(19) United States
(12) Patent Application Publication
(10) Pub. No.: US 2024/0390591 A1
(43) Pub. Date: Nov. 28, 2024

(54) PRE-FILLED SYRINGE WITH OPTIMIZED STOPPER PLACEMENT

(71) Applicant: Janssen Pharmaceutica NV, Beerse (BE)

(72) Inventors: Ignace Wallaert, Westmalle (BE); Marian van der Veen, Eindhoven (NL); Joris DeWolf, Mechelen (BE)

(21) Appl. No.: 18/651,946

(22) Filed: May 1, 2024

Related U.S. Application Data
(63) Continuation of application No. 17/458,878, filed on Aug. 27, 2021, now abandoned.
(60) Provisional application No. 63/073,777, filed on Sep. 2, 2020.

(51) Int. Cl. A61M 5/31; A61K 31/519; A61M 5/315; A61M 5/32

(52) U.S. Cl. CPC A61M 5/3134; A61K 31/519; A61M 5/31505; A61M 5/3202; A61M 5/3293; A61M 2005/3131

(57) ABSTRACT

A pre-filled syringe includes a barrel with a medicament and a stopper within the barrel. The stopper has at least two adjacent sealing rings and a trim ring. The syringe has a head space distance between a distal-most surface of the stopper and the surface of the medicament. The head space (a) provides sufficient space to optimize reconstituting a drug suspension by shaking or agitating the drug suspension, and (b) minimizes frothing and/or clogging of the reconstituted drug suspension in the barrel. Also, a kit includes the syringe, a plunger, a syringe cap, and a needle assembly. Further, a method for stoppering the syringe includes determining the head space distance.

### CROSS REFERENCE TO RELATED APPLICATIONS

This application is a continuation of U.S. application Ser. No. 17/458,878, filed on Aug. 27, 2021 which claims priority from U.S. Provisional Application 63/073,777, filed on Sep. 2, 2020, all of which are incorporated by reference herein in their entirety.

### FIELD

The present disclosure relates generally to syringes and syringe kits, and related methods.

### BACKGROUND

A significant number of people are given medications to treat a variety of limited and ongoing medical conditions, and a large number of these medications are delivered into a user's body through a syringe. Numerous treatments also require administration of these medications via syringe according to strict dosing regimens. Additionally, many of these medications are shipped as a suspension or in other forms in a pre-filled syringe, and the medication must be mixed, for example, by shaking the syringe, immediately prior to administration. In some instances, if the medication is not properly mixed and administered to the user, there can be risks of harm to the patient due to a lack of effective treatment.

As such, providing a pre-filled syringe configured to withstand storage conditions and ensure proper mixing of medications shipped in a syringe can provide greater convenience and efficacy of treatment.

Accordingly, there remains a need for improved pre-filled syringes.

### SUMMARY

In general, pre-filled syringes, corresponding kits, systems, and methods for using the same are provided.

A pre-filled syringe includes a barrel storing medicament and a stopper with optimized head space (i.e., a distance or volume between a surface of a medicament and an opposed surface of a stopper). The head space (a) provides sufficient space to optimize reconstituting a drug suspension by shaking or agitating the drug suspension, and (b) minimizes frothing and/or clogging of the reconstituted drug suspension in the barrel.

The pre-filled syringe may include a barrel having a first, proximal end and a second, distal end with a medicament disposed therein. The pre-filled syringe may include a stopper disposed within the barrel adjacent the first end of the barrel, the stopper having a body with a distal end and a proximal end and having an outer sidewall extending therebetween, the outer sidewall having at least two adjacent scaling rings extending radially from a minor diameter of the body to a major diameter of the body. The pre-filled syringe may include a trim ring extending radially to the major diameter of the body, the trim ring being proximal to the proximal-most sealing ring. The pre-filled syringe may have a head space distance between a distal-most surface of the stopper and the surface of the medicament. The head space may (a) provides sufficient space to optimize reconstituting a drug suspension by shaking or agitating the drug suspension, and (b) minimizes frothing and/or clogging of the reconstituted drug suspension in the barrel. The pre-filled syringe may have a stopper height that is determined by a distance from a proximal-most surface of the syringe to a surface of the medicament less the sum of a length of the stopper and the head space.

For a presentation of the medicament having a volume of about 3.5 mL, a ratio of a volume of the target head space plus a medicament volume to the medicament volume may be greater than about 1.00 and less than about 2.34. For the presentation having a volume of about 5 mL, a ratio of a volume of the target head space plus a medicament volume to the medicament volume may be greater than about 0.70 and less than about 1.64.

A ratio of a target head space distance to a target volume of a presentation of the medicament may be greater than about 0.60 mm/mL and less than about 2.00 mm/mL. For the presentation having a volume of about 3.5 mL, the ratio of the target head space distance to the target volume of the presentation of the medicament may be greater than about 0.86 mm/mL and less than about 2.00 mm/mL. For the presentation having a volume of about 5 mL, the ratio of the target head space distance to the target volume of the presentation of the medicament may be greater than about 0.60 mm/mL and less than about 1.40 mm/mL.

A target head space distance between a surface of the medicament and a proximal end of the stopper may be greater than about 3 mm and less than about 7 mm, i.e., about 5 mm±2 mm. The target head space distance between the surface of the medicament and the proximal end of the stopper may be greater than about 4 mm and less than about 6 mm, i.e., about 5 mm±1 mm. The target head space distance between the surface of the medicament and the proximal end of the stopper may be about 5 mm.

The major diameter of the body may be about 117% to about 130% larger than a length of the body in an axial direction of the syringe. The major diameter of the body may be about 111% to about 118% larger than the minor diameter of the body. The body may have a length in an axial direction of the syringe of about 10.3 mm±0.4 mm.

At least two of the adjacent sealing rings may be provided a spaced distance apart from each other in the axial direction of the syringe. The at least two sealing rings may include at least three sealing rings. The at least three sealing rings may consist of three sealing rings. The spaced distance may be about 2.9 mm. A portion of a radial end of at least one of the adjacent sealing rings has a circular cross section with a radius of about 0.7 mm. The major diameter of the body may be about 12.7 mm±0.15 mm. The minor diameter of the body may be about 11.1 mm±0.15 mm.

The pre-filled syringe may further include a plunger rod attached to the stopper and extending out through the first open end of the barrel.

The medicament may be a suspension of paliperidone palmitate. The suspension of paliperidone palmitate may have a concentration of about 200 mg eq./mL (as an actual salt, paliperidone palmitate may be provided at a concentration of about 312 mg/mL). The suspension of paliperidone palmitate may be used for a six month treatment.

The pre-filled syringe may be configured for use with any one of an 18 gauge, 19 gauge, 20 gauge, 21 gauge, 22 gauge and 23 gauge cannula. In some embodiments, the pre-filled syringe may be configured for use with a 20 gauge or 22 gauge cannula.

A kit may include a pre-filled syringe. The pre-filled syringe may include a barrel having a first, proximal end and a second, distal end with a medicament disposed therein. The pre-filled syringe may include a connection at the distal end of the barrel. The pre-filled syringe may include a stopper disposed within the barrel adjacent the first end of the barrel. The stopper may have a body with a distal end and a proximal end. The stopper may have an outer sidewall extending therebetween. The outer sidewall may have at least two adjacent scaling rings extending radially from a minor diameter of the body to a major diameter of the body. The outer sidewall may have a trim ring extending radially to the major diameter of the body. The trim ring may be proximal to the proximal-most sealing ring. The pre-filled syringe may have a stopper height that is determined by a distance from a proximal-most surface of the syringe to a surface of the medicament less the sum of a length of the stopper and the head space. The pre-filled syringe may have a head space distance between a distal-most surface of the stopper and the surface of the medicament. The head space may (a) provide sufficient space to optimize reconstituting a drug suspension by shaking or agitating the drug suspension, and (b) minimize frothing and/or clogging of the reconstituted drug suspension in the barrel.

The kit may further include a plunger configured for removable engagement with the proximal end of the stopper. The kit may further include a syringe cap configured to engage the connection and close the barrel. The kit may further include a needle assembly. The needle assembly may include a pouch containing a cannula sheath containing a cannula. The cannula sheath may include a hub. The hub may be configured to engage with the connector element. The hub may be configured to permit selective delivery of the medicament through the cannula.

For the presentation having a volume of about 3.5 mL, a ratio of a volume of the target head space plus a medicament volume to the medicament volume may be greater than about 1.00 and less than about 2.34. For the presentation having a volume of about 5 mL, a ratio of a volume of the target head space plus a medicament volume to the medicament volume may be greater than about 0.70 and less than about 1.64.

A ratio of a target head space distance to a target volume of a presentation of the medicament may be greater than about 0.60 mm/mL and less than about 2.00 mm/mL. For the presentation having a volume of about 3.5 mL, the ratio of the target head space distance to the target volume of the presentation of the medicament may be greater than about 0.86 mm/mL and less than about 2.00 mm/mL. For the presentation having a volume of about 5 mL, the ratio of the target head space distance to the target volume of the presentation of the medicament may be greater than about 0.60 mm/mL and less than about 1.40 mm/mL.

A target head space distance between a surface of the medicament and a proximal end of the stopper may be greater than about 3 mm and less than about 7 mm. The target head space distance between the surface of the medicament and the proximal end of the stopper may be greater than about 4 mm and less than about 6 mm. The target head space distance between the surface of the medicament and the proximal end of the stopper may be about 5 mm.

The major diameter of the body may be about 117% to about 130% larger than a length of the body in an axial direction of the syringe. The major diameter of the body may be about 111% to about 118% larger than the minor diameter of the body. The body may have a length in an axial direction of about 10.3 mm±0.4 mm.

At least two of the adjacent sealing rings may be provided a spaced distance apart from each other in the axial direction of the syringe. The at least two sealing rings may include at least three sealing rings. The at least three sealing rings may consist of three sealing rings. The spaced distance may be about 2.9 mm. A portion of a radial end of at least one of the adjacent sealing rings has a circular cross section with a radius of about 0.7 mm.

The major diameter of the body may be about 12.7 mm±0.15 mm. The minor diameter of the body may be about 11.1 mm±0.15 mm.

The kit may further include a plunger rod attached to the stopper and extending out through the first open end of the barrel.

The medicament may be a suspension of paliperidone palmitate. The suspension of paliperidone palmitate may have a concentration of about 200 mg eq./mL (as an actual salt, paliperidone palmitate may be provided at a concentration of about 312 mg/mL). The suspension of paliperidone palmitate is used for a six month treatment.

The cannula may be any one of an 18 gauge, 19 gauge, 20 gauge, 21 gauge, 22 gauge and 23 gauge cannula.

A method includes providing a pre-filled syringe. The pre-filled syringe may include a barrel having a first, proximal end, a second, distal end, and a connection at the distal end. The method may include providing a stopper. The stopper may have a body with a distal end and a proximal end and having an outer sidewall extending therebetween. The outer sidewall may have at least two adjacent sealing rings extending radially from a minor diameter of the body to a major diameter of the body. The outer sidewall may have a trim ring extending radially to the major diameter of the body. The trim ring may be proximal to the proximal-most scaling ring. The method may include providing a cap and/or a cannula assembly. The method may include closing the distal end of the barrel with the cap or the cannula assembly via the connection. The method may include disposing a medicament via an opening in the proximal end of the barrel. The method may include disposing the stopper within the barrel adjacent the proximal end of the barrel. The method may include determining a head space distance from a proximal-most surface of the syringe to a surface of the medicament. The head space may (a) provide sufficient space to optimize reconstituting a drug suspension by shaking or agitating the drug suspension, and (b) minimize frothing and/or clogging of the reconstituted drug suspension in the barrel.

For the presentation having a volume of about 3.5 mL, a ratio of a volume of the target head space plus a medicament volume to the medicament volume may be greater than about 1.00 and less than about 2.34. For the presentation having a volume of about 5 mL, a ratio of a volume of the target head space plus a medicament volume to the medicament volume may be greater than about 0.70 and less than about 1.64.

A ratio of a target head space distance to a target volume of a presentation of the medicament may be greater than about 0.60 mm/mL and less than about 2.00 mm/mL. For the presentation having a volume of about 3.5 mL, the ratio of the target head space distance to the target volume of the presentation of the medicament may be greater than about 0.86 mm/mL and less than about 2.00 mm/mL. For the presentation having a volume of about 5 mL, the ratio of the target head space distance to the target volume of the presentation of the medicament may be greater than about 0.60 mm/mL and less than about 1.40 mm/mL.

A target head space distance between a surface of the medicament and a proximal end of the stopper may be greater than about 3 mm and less than about 7 mm. The target head space distance between the surface of the medicament and the proximal end of the stopper may be greater than about 4 mm and less than about 6 mm. The target head space distance between the surface of the medicament and the proximal end of the stopper may be about 5 mm.

The major diameter of the body may be about 117% to about 130% larger than a length of the body in an axial direction of the syringe. The major diameter of the body may be about 111% to about 118% larger than the minor diameter of the body. The body may have a length in an axial direction of the syringe of about 10.3 mm±0.4 mm. At least two of the adjacent sealing rings may be provided a spaced distance apart from each other in the axial direction of the syringe. The at least two sealing rings may include at least three sealing rings. The at least three sealing rings may consist of three sealing rings. The spaced distance may be about 2.9 mm. A portion of a radial end of at least one of the adjacent sealing rings has a circular cross section with a radius of about 0.7 mm.

The major diameter of the body may be about 12.7 mm±0.15 mm. The minor diameter of the body may be about 11.1 mm±0.15 mm.

The method may further include attaching a plunger rod to the stopper with the plunger rod extending out through the first open end of the barrel.

The head space distance may be confirmed with a camera. Specifically, the camera may detect a stopper height and the head space may be calculated from the stopper height.

The medicament may be a suspension of paliperidone palmitate. The suspension of paliperidone palmitate may have a concentration of about 200 mg eq./mL (as an actual salt, paliperidone palmitate may be provided at a concentration of about 312 mg/mL). The suspension of paliperidone palmitate is used for a six month treatment.

The method may further include providing any one of an 18 gauge, 19 gauge, 20 gauge, 21 gauge, 22 gauge and 23 gauge cannula.

### DESCRIPTION OF DRAWINGS

This invention will be more fully understood from the following detailed description taken in conjunction with the accompanying drawings, in which:

FIG. 1A is a side view of a syringe pre-filled with a medicament after placement of a stopper within a proximal end of the syringe with a cap attached to a distal end of the pre-filled syringe and a plunger and finger flange attached to a proximal end of the pre-filled syringe according to an exemplary embodiment;

FIG. 1B is a side view of a needle pouch, which contains a needle sheath, which covers a needle and which is configured for attachment to a Luer connection on a distal end of the pre-filled syringe according to an exemplary embodiment;

FIG. 2A is a partial cross-sectional side view and a partial side exterior view of a stopper according to an exemplary embodiment with the distal end up and the proximal end down;

FIG. 2B is an end view of the proximal end of the stopper;

FIG. 2C is an end view of the distal end of the stopper;

FIG. 2D is a partial cross-sectional side view of detail 2D of FIG. 2A showing an interior helically threaded surface of the stopper;

FIG. 2E is a perspective view of the stopper including the proximal end;

FIG. 3 is a schematic side view of a syringe pre-filled with medicament after placement of a stopper within a proximal end of the syringe according to an exemplary embodiment;

FIG. 4A is a side cross-sectional view of a stopper according to an exemplary embodiment;

FIG. 4B is a side cross-sectional view of a stopper according to another exemplary embodiment;

FIG. 5A is a side view of a syringe after placement of a stopper within a proximal end of the syringe beside a digital depth meter for determining a distance from a top edge of the syringe to an internal wall within the stopper adjacent to the distal end of the stopper;

FIG. 5B is a side view of the syringe of FIG. 5A with a distal end of the digital depth meter placed inside the syringe and in contact with the internal wall within the stopper adjacent to the distal end of the stopper;

FIG. 6 is an image of a side view of a proximal end of a syringe used to determine a distance between a proximal end of a flange of the syringe and a proximal-most surface of a stopper and a distance between a top surface of a liquid within the syringe and the proximal end of the flange of the syringe according to an exemplary embodiment;

FIG. 7A is an image of a side of a pre-filled syringe according to an exemplary embodiment with a relatively small head space between a liquid in the syringe and a distal end of a stopper and with a portion of the liquid in the syringe making and maintaining contact with the stopper;

FIG. 7B is an image of a side of a pre-filled syringe according to an exemplary embodiment with a relatively large head space and little if any of the liquid in the syringe making and maintaining contact with the stopper;

FIG. 8A is a side view of a pre-filled syringe in a first stage with the syringe filled with medicament prior to placement of a stopper on a proximal end of the pre-filled syringe, and with a cannula attached to a distal end of the pre-filled syringe according to an exemplary embodiment;

FIG. 8B is a side view of the pre-filled syringe of FIG. 8A in a second stage with a stopper to-be-placed within the syringe using a temporary sheath inserted into and engaged with an interior of the syringe, and with a temporary push rod engaged with a proximal end of the stopper near a proximal end of the temporary sheath;

FIG. 8C is a side view of the pre-filled syringe of FIG. 8A in a third stage with the temporary push rod extended to place the stopper at or near the end of the temporary sheath;

FIG. 8D is a side view of the pre-filled syringe of FIG. 8A in a fourth stage with the temporary push rod still extended in contact with the stopper and with the temporary sheath being retracted from the syringe;

FIG. 8E is a side view of the pre-filled syringe of FIG. 8A in a fifth stage with the temporary push rod and the temporary sheath fully retracted from the syringe leaving the stopper in place within the syringe;

FIG. 8F is a side view of the pre-filled syringe of FIG. 8A in a sixth stage with the stopper in place within the syringe;

FIG. 9 is a chart with the results of an experiment including Trials 1-8, inclusive, each with a stopper according to an exemplary embodiment in 5 mL presentations, i.e., experimental trials utilizing the P8790TR-stopper with overfill syringes in the 5 mL presentation;

FIG. 10A is an image similar to FIG. 6 for Trial 1 of 8, the results of which are illustrated in FIG. 9;

FIG. 10B is an image for Trial 2 of 8;

FIG. 10C is an image for Trial 3 of 8;

FIG. 10D is an image for Trial 4 of 8;

FIG. 10E is an image for Trial 5 of 8;

FIG. 10F is an image for Trial 6 of 8;

FIG. 10G is an image for Trial 7 of 8;

FIG. 10H is an image for Trial 8 of 8; and

FIG. 11 is a flow chart illustrating steps of a method of providing a pre-filled syringe according to an exemplary embodiment.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed kits, systems, devices, and methods, such dimensions are not intended to limit the types of shapes that can be used in conjunction with such kits, systems, devices, and methods. A person skilled in the art will recognize that an equivalent to such linear and circular dimensions can easily be determined for any geometric shape. Sizes and shapes of the kits, systems and devices, and the components thereof, can depend at least on the anatomy of the subject in which the kits, systems and devices will be used, the size and shape of components with which the kits, systems and devices will be used, and the methods and procedures in which the kits, systems and devices will be used. Like reference symbols in the various drawings indicate like elements.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Various exemplary pre-filled syringes with optimized stopper placement, corresponding kits, systems, kits, and methods for using the same are provided. The present disclosure generally relates to pre-filled syringes with optimized stopper placement. The syringes disclosed herein are pre-filled with a medicament, typically one that is in the form of a liquid suspension.

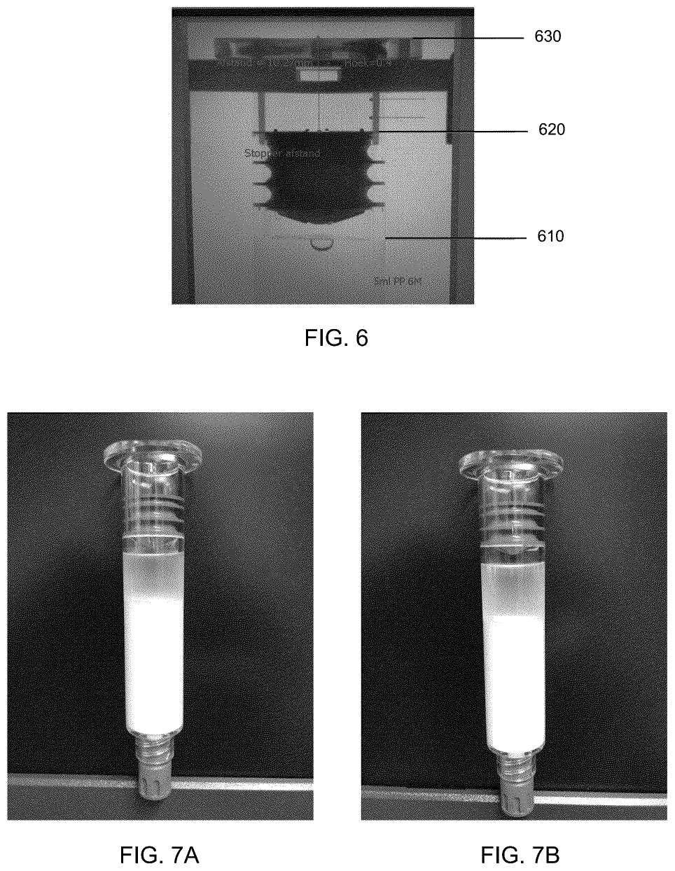

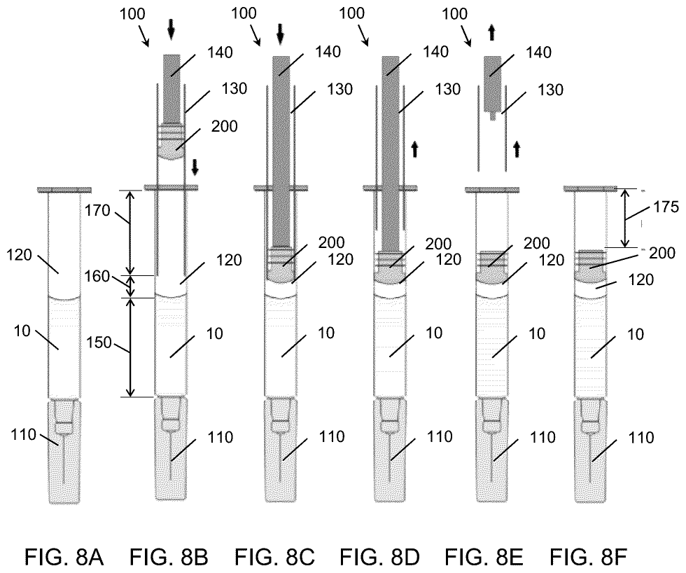

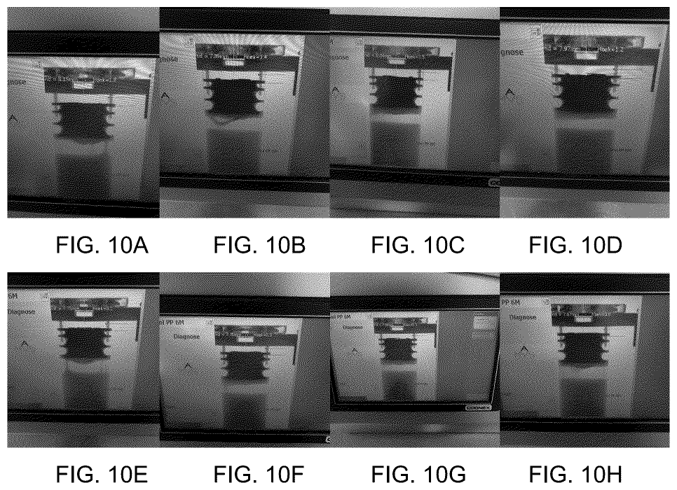

A “medicament” as used herein refers to a therapeutic agent (a drug, a biologic, a biological material, etc.) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. Non-limiting examples of suitable medicaments include paliperidone palmitate, such as paliperidone palmitate equivalent to 200 mg/mL extended release suspension for injection. A pre-filled syringe assembly may have a stopper height within a syringe barrel to optimize a head space between a surface of the liquid or medicament within the syringe barrel and a distal end of the stopper. As is known in the art, a liquid such as water may form a concave meniscus with respect to an internal wall of a container. It is understood herein that a distance to a surface of a liquid is measured from a lowermost point of a concave meniscus or an uppermost point of a convex meniscus. In some embodiments, the meniscus may be flat or nearly flat. For example, in FIG. 6 (an example using water), the meniscus appears to be flat or nearly flat; in FIGS. 7A and 7B (an example with medicament in suspension before shaking), the meniscus is slightly concave; as depicted in FIGS. 8A to 8F, the meniscus is concave; and in FIGS. 10A to 10H (examples with medicament in suspension after shaking), the meniscus is flat or nearly flat. Optimization of head space provides for (a) sufficient space to optimize reconstituting a drug suspension by shaking or agitating the suspension, and (b) minimized “frothing” and/or “clogging” of the reconstituted drug suspension in the syringe barrel. That is, too much head space may contribute to excess and undesirable frothing of the reconstituted drug suspension. On the other hand, too little head space may contribute to improper mixing of the drug suspension, and thus the presence of relatively large particles in the suspension, which can clog the delivery cannula.

In addition to the optimized stopper height and head space, the stopper is designed to provide optimized engagement between the stopper and an inner surface of the syringe barrel. The “break loose” force, i.e., the force needed to effect initial movement of the stopper during injection, is sufficient to retain the position of the stopper during storage and/or shipping such that the stopper position, and thus the head space, is not inadvertently altered. At the same time, the break loose force is not so great that a high force is required to move the stopper during an injection procedure. In addition to making injection difficult and/or uncomfortable, an excessively high break loose force may reduce the ability of a user to control an injection. In one aspect the optimized engagement between the stopper and the syringe barrel may be provided by including in the stopper a modified proximal ring or “trimming ring,” as explained below. In some exemplary embodiments, at least two sealing rings are provided. The at least two sealing rings may include at least three sealing rings. The at least three sealing rings may consist of three sealing rings. In some exemplary embodiments, the three sealing rings in combination with the trim ring provided a synergistic combination of desirable resistance and movement during administration of the medicament to a patient in need of treatment while also ensuring secure placement of the stopper within the syringe barrel, particularly during shipment where external forces might impact a shipping container containing one or more filled syringes. However, the stopper is not limited to this configuration. The stopper may have less or more than three scaling rings. The stopper may or may not have the trim ring.

In an exemplary embodiment, relative to the dimensions of known stoppers for syringes, the improved stopper design may include one or more of the following features: relatively thick ribs, rings or other protrusions (measured in an axial direction of the stopper), a relatively long stopper length (measured in the axial direction of the stopper), a relatively large stopper diameter, an extra rib, ring or other protrusion (e.g., a proximal or trimming ring) on a proximal end of the stopper. In one aspect, the trim ring has a diameter slightly larger than or approximately equal to that of the ribs, rings or other protrusions, a cross section that is squared-off (i.e., not circular or rounded), and relatively large radius ribs, rings or other protrusions (in side cross section) to provide greater contact surface with an interior of the syringe body.

The present disclosure has applicability to a multitude of medicaments that can be injected into a patient, particularly those in the form of a liquid suspension that must be reconstituted, such as by agitation, prior to injection. As noted above, paliperidone palmitate is one example of such a medicament. In some embodiments, the paliperidone palmitate is equivalent to about 200 mg/mL extended release suspension for injection that is intended to be a six month dosage. For the about 3.5 mL and about 5 mL presentations of this medicament, it was determined, for example, that a head space of greater than about 3 mm and less than about 7 mm minimized both frothing and clogging problems. In some embodiments, the head space is greater than about 4 mm and less than about 6 mm. In other embodiments, the head space is about 4 mm. In some embodiments, the head space is about 5 mm.

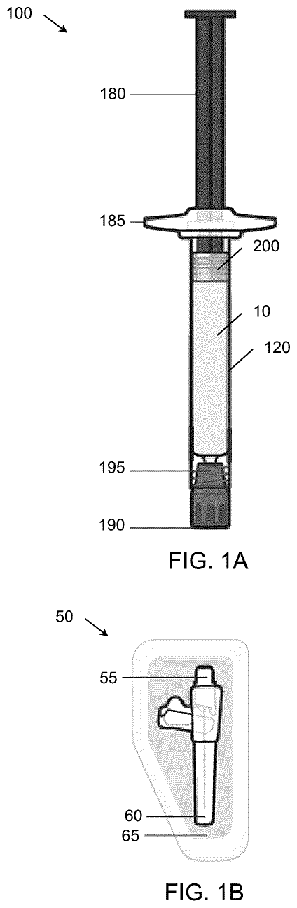

As shown in FIG. 1A, a pre-filled syringe assembly 100 includes a syringe barrel 120 filled with a liquid or medicament 10. A stopper 200 seals the syringe assembly 100 on a proximal end adjacent to finger flange 185. Specifically, as shown in FIG. 1A, the pre-filled syringe assembly 100 is pre-filled with the medicament 10 and stoppered with the stopper 200 that is within the proximal end of the pre-filled syringe barrel 120. At the distal end, the pre-filled syringe barrel 120 is closed with a cap 190 e.g., via a Luer connection 195 that will facilitate connection of the syringe to an injection needle. While FIG. 1A illustrates a plunger 180 attached to a proximal end of the pre-filled syringe barrel 120 to facilitate manual injection of the medicament 10, it is understood that the plunger 180 can be removably attached to the stopper and that such attachment may take place after storage and before use of the syringe to inject the medicament into a patient. The pre-filled syringe assembly 100 may be provided in a kit that contains the pre-filled syringe assembly 100 as that shown in FIG. 1A, i.e., with the cap 190 in place via the Luer connection 195 (with the plunger 180 pre-attached or available for attachment to the pre-filled syringe assembly 100 before use) and a needle assembly 50 (sealed in a sterile package) such as that shown in FIG. 1B. The cap 190 can be removed from the assembly 100, the needle assembly 50 can be removed from the sterile package, and the needle assembly 50 can be attached to the assembly 100 via the Luer connection 195 prior to injection.

As shown in FIG. 1B, the needle assembly 50 of the kit includes a needle pouch 65, which contains a needle sheath 60, which covers a needle or cannula. The needle sheath 60 has a hub 55, and the hub 55 is configured for attachment to the Luer connection 195 on the distal end of the pre-filled syringe barrel 120.

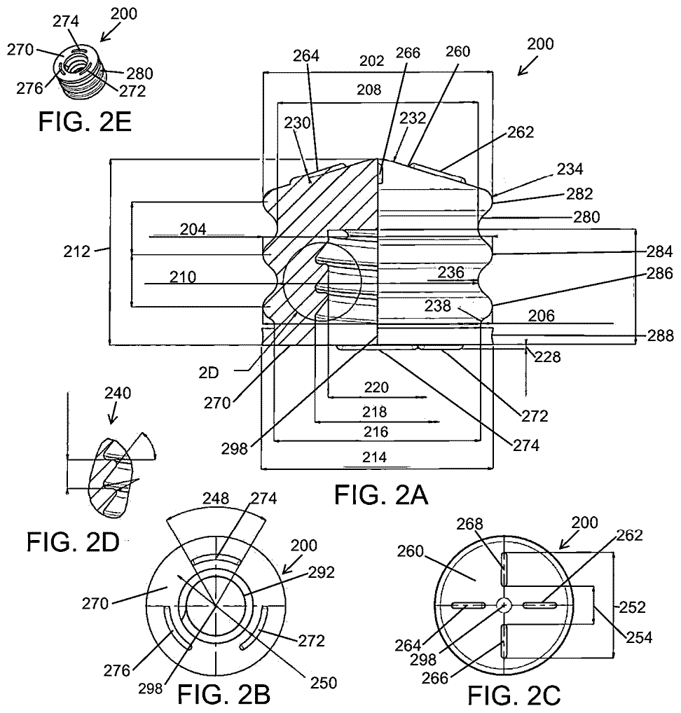

An optimized stopper is provided to ensure secure engagement between the stopper and an inner surface of the syringe barrel. For example, as shown in FIGS. 2A to 2E, inclusive, the stopper 200 has a generally hollow cylindrical shape. An outer side surface 280 of the stopper 200 has a plurality of radially outwardly extending ribs, rings or other protrusions (see FIGS. 2A and 2E).

In one exemplary embodiment, for use with about 3.5 mL and about 5.0 mL fill volumes, a 5 mL Schott TopPac syringe may be used. An internal diameter of the Schott TopPac syringe is about 12.2 mm±0.1 mm.

The stopper 200 includes a first rib, ring or other protrusion 282, a second rib, ring or other protrusion 284, and a third rib, ring or other protrusion 286. The first rib, ring or other protrusion 282 is sized such that the major diameter 202 of the stopper measured at the first rib, ring or other protrusion 282 is about 12.7 mm±0.15 mm. Similarly, each of the major diameter 204 of the stopper 200 measured at the second rib, ring or other protrusion 284, and the major diameter of the stopper 200 measured at the third rib, ring or other protrusion 286 is about 12.7 mm±0.15 mm. The minor diameter of the stopper 200, measured in a valley or trough between the first rib, ring or other protrusion 282 and the second rib, ring or protrusion 284, and between the second rib, ring or protrusion 284 and the third rib, ring or protrusion 286, is about 11.1 mm±0.15 mm. A total length 212 of the stopper 200 measured from a distal tip (the portion of the stopper 200 at or near a center 298 of the stopper 200 as seen in FIG. 2C) to a proximal end face 270 (exclusive of any projections, described below) is about 10.3 mm±0.4 mm.

The distal tip of the stopper may have a shape configured to prevent liquid from collecting on the stopper. For example, the distal tip may have a somewhat arced surface when viewed through a side cross section such as in FIG. 2A, and the curvature may correspond with a circle having a radius at arrow 232 of about 2.3 mm. Each of the first rib, ring or other protrusion 282, the second rib, ring or other protrusion 284, and the third rib, ring or protrusion 286 may have a radially outermost portion with a circular curvature when viewed through a side cross section such as in FIG. 2A, and the curvature may correspond with a circle having a radius at arrow 234 of about 0.7 mm. In the valley or crevice between the first rib, ring or other protrusion 282 and the second rib, ring or other protrusion 284 and between the second rib, ring or other protrusion 284 and the third rib, ring or other protrusion 286, a radially innermost portion may have a circular curvature when viewed through a side cross section such as in FIG. 2A, and the curvature may correspond with a circle having a radius at arrow 236 of about 0.9 mm.

To further ensure secure engagement between the stopper and an inner surface of the syringe barrel, the stopper 200 includes a proximal or trimming ring 288. The ring 288 may be trimmed out of a molded sheet containing an array of stoppers. For example, when the stopper 200 is stamped out of a thermoformed sheet containing multiple stoppers, relatively straight sides of the trimming ring 288 may result. In addition to holding force provided by the rings 282, 284, 286, the trimming ring 288 may function to provide additional holding force when inserted into the syringe barrel 120. An outside diameter 214 of the stopper 200 measured at the trim ring 288 may be about 12.8 mm, which is slightly larger than the major diameter 202 of the stopper 200 measured at each of ribs, rings or other protrusions 282, 284, and 286. In some embodiments, the outside diameter of the stopper 200 measured at the trim ring 288 may be about the same as the major diameter 202 of the stopper 200 measured at each of ribs, rings or other protrusions 282, 284, and 286. In a valley or crevice between the third rib, ring or other protrusion 286 and the trim ring 288, an outside diameter 216 may be about 11.4 mm. In the valley or crevice between the third rib, ring or other protrusion 286 and the trim ring 288, a radially innermost portion may have a circular curvature when viewed through a side cross section such as shown in FIG. 2A, and the curvature may correspond with a circle having a radius at arrow 238 of about 0.4 mm.

As shown in FIGS. 2A, 2D, and 2E, stopper 200 has an internally threaded cavity (best seen in FIG. 2A, detail at FIG. 2D) that is configured to receive and allow mating of plunger rod 180 in threaded engagement therewith. As illustrated in detail 240 (FIG. 2D), the cavity includes a helically threaded inner surface. A person skilled in the art will understand that the threaded inner surface of cavity can have any suitable thread form that is complimentary to threads formed on a portion of the plunger rod 180 that will facilitate secure mating of the plunger rod 180 to stopper 200 so as to enable injection to be effected. Also, in lieu of a threaded connection, any suitable connection type may be utilized.

As shown in FIGS. 2B and 2E, in one embodiment a plurality of projections project proximally from the proximal end face 270. For example, the proximal end face 270 includes a first proximal projection 272, a second proximal projection 274 and a third proximal projection 276. While the size and shape of projections 272, 274, 276 can vary, in one embodiment each of these projections has a height of about 0.25 mm. Further, in the illustrated embodiment each of projections 272,274, 276 has a curved shape with two rounded ends as shown in FIG. 2B, and the curved shape can correspond with an arc through an angle 248 of about 60 degrees. The projections 272, 274, 276 may function to engage with the plunger 180. For example, the projections 272, 274, 276 may provide compressive resistance or a locking mechanism when engaged with a distal end of the plunger 180. The projections 272, 274, 276 are optional.

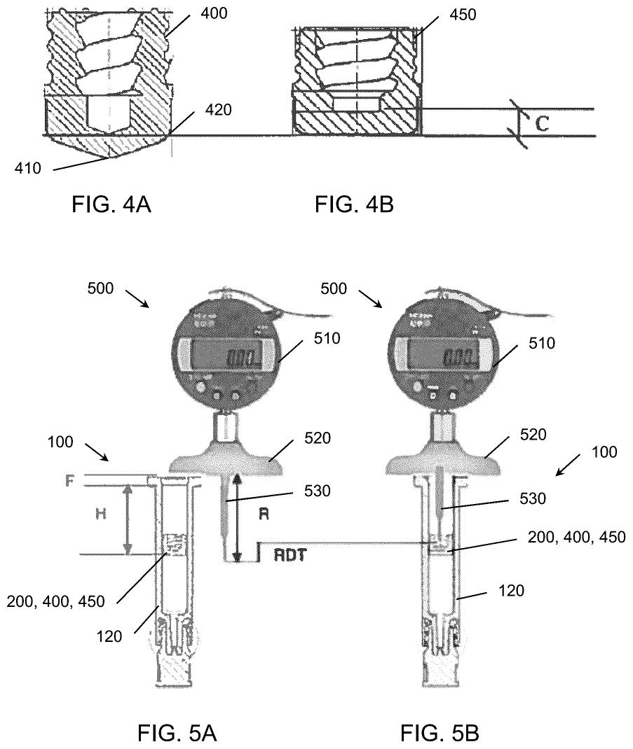

As shown in FIG. 2C, a plurality of distal projections are provided on a distal end of the stopper 200. For example, the distal end surface 260 includes a first distal projection 262, a second distal projection 264, a third distal projection 266 and a fourth distal projection 268. While the size and shape of the distal projections 262, 264, 266, 268 can vary, in one embodiment each of the distal projections has a height 230 (FIG. 2A) of about 0.25 mm. Further, in the illustrated embodiment, each of the distal projections 262, 264, 266, 268 has a generally linear shape with two rounded ends as shown in FIG. 2C. A distance 252 in a radial direction from an outermost rounded end of one projection (e.g., 262, 264, 266, 268) to an outermost rounded end of an opposite projection (e.g., 264, 262, 268, 266, respectively) is about 9.7 mm. A distance 254 in a radial direction from an innermost rounded end of one projection (e.g., 262, 264, 266, 268) to an innermost rounded end of an opposite projection (e.g., 264, 262, 268, 266, respectively) is about 3.5 mm. Each of the first distal projection 262, the second distal projection 264, the third distal projection 266 and the fourth distal projection 268 is arranged at approximately equal 90 degree intervals as shown in FIG. 2C. In one embodiment, the distal end surface 260 may include a frustoconical shape as shown in FIGS. 2A and 4A). Alternatively, the distal end surface 260 may be relatively flat as shown in FIG. 4B). The projections 262, 264, 266, 268 may function as a drip edge of the stopper 200. For example, after shaking the filled assembly 100, the projections 262, 264, 266, 268 may facilitate collection of medicament that sticks to the stopper 200 into drops, which, through gravity, return to the main reservoir of medicament in the assembly 100. The projections 262, 264, 266, 268 are optional.

Precise placement of the stopper within the syringe body is desirable for all of the reasons discussed above. Having described exemplary assemblies and stopper dimensions, optimized positioning of a stopper within a syringe body and exemplary procedures for determining such optimized positioning of a stopper within a syringe body are provided below.

As explained below, Example 1 is directed to one method of determining a stopper height for the stopper 200 relative to the syringe barrel 120 according to an exemplary embodiment, particularly a method to assure that an about 5 mm±1 mm head space is provided in syringes for paliperidone palmitate equivalent to about 200 mg/mL injectable suspension, typically used for a six month treatment (hereinafter “PP6M”) for two fill volumes about 3.5 mL and about 5.0 mL; Example 2 is directed to another method of determining a stopper height for the stopper 200 relative to the syringe barrel 120 according to an exemplary embodiment; Example 3 is directed to a method of determining head space for the PP6M medicament; and Example 4 is directed to a method of determining a stopper position for overfill syringes in the about 5 mL presentation.

### Example 1: Determination of Stopper Height Position with 5 mm Head Space

The stopper height measurement is an In Process Control (“IPC”) during the filling/stoppering operation. The target stopper height position assures an about 5 mm±1 mm head space in syringes for paliperidone palmitate equivalent to about 200 mg/mL injectable suspension (hereinafter “PP6M”) for fill volumes of about 3.5 mL and about 5.0 mL. Stopper height position is determined by subtracting the height of the stopper and the distance corresponding with a target head space from a distance from a meniscus of the liquid to the proximal end of the syringe.

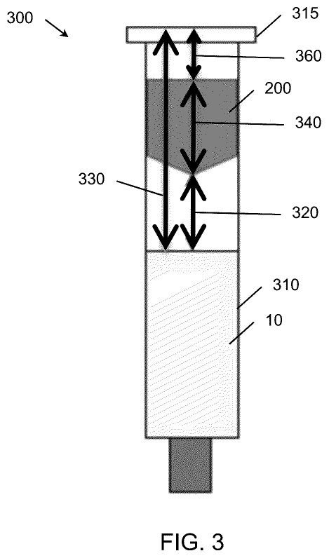

FIG. 3 depicts an exemplary embodiment of a pre-filled syringe assembly 300 including a syringe 310 filled with a liquid or medicament 10. For liquids 10 surface having a concave meniscus shape, the lowest point or bottom of the meniscus is taken for the measurement. Head space 320 is the distance from the liquid 10 surface to the lowest point of the stopper 200. Stopper length 340 is the length of the stopper 200 in the syringe 310. (The length 340 of the stopper 200 may change prior to insertion and after insertion and may vary depending on the amount of force used to insert the stopper 200.) The stopper length 340 may also be expressed as the distance from the lowest point of the stopper 200 to the top (see, and compare, the total length 212 of the stopper 200 of FIG. 2A). Stopper height position 360 is the distance from the top of the syringe flange 315 to the top of the stopper 200. The stopper height position 360 for the about 3.5 and about 5 mL filled PP6M syringe is determined by taking the distance 330 from the top of the syringe flange 315 to liquid 10 surface minus the stopper length 340 in the syringe 340 and the head space 320. That is:

Stopper height position (mm) 360=a−(b+c), where
a=height 330 from a top of the syringe to a liquid surface
b=head space (±5 mm) 320
c=stopper length (mm) 340

Head space may also be expressed in terms of a ratio or percentage of a total volume below the stopper (i.e., volume of medicament and head space) relative to a volume of liquid or medicament in the syringe.

Study: Five (5) syringes 310 of both fill volumes (about 3.5 mL and about 5 mL) were filled with purified water at target fill volume (about 3.616 mL and about 5.104 mL); and a stopper 200 was manually placed with a head space 320 of about 5 mm.

Measurement and calculations: The distance 330 from the top of the syringe flange to the surface of the liquid, the head space 320 and the stopper length 340 of the 10 syringes were measured (see, Table 1) and the stopper height position 360 was calculated.

The measured stopper length 340 in the syringe 310 deviates from the technical drawing (see, length 212 in FIG. 2A) due to stretching of the stopper 200 during stoppering. The length of the stopper according to the technical drawing is about 10.3 mm±0.4. The average length after placement is about 11.0 mm with a standard deviation (Stdev) of 0.02 mm.

The calculations were performed using the stopper length of about 10.3 mm and about 11.0 mm. Based on the results, the data from the calculated stopper height (calculated with 11 mm) appears to be more in line with the measured stopper height data.

TABLE 1
Measured head space, height from top of syringe flange to liquid surface and
stopper height position and calculated stopper height position. (While the
stopper height is 10.3 +/− 0.4 mm, the stopper height used in the
calculation below is 11.0 mm because the stopper is squeezed in the barrel,
causing it to be slightly longer than when it is not placed in a syringe.)

| | | | | | Calculated | | | |
|---|---|---|---|---|---|---|---|---|
| | | | | | Stopper | | Stopper | |
| | | Measured | | | height | | height | |
| | | | Height | | position | | position | |
| | | | from top | Stopper | (mm) | | (mm) | |
| | | Head | to top | height | (stopper | | (stopper | |
| | | space | liquid | position | length | | length | |
| | Syringe | (mm) | (mm) | (mm) | 11.0 mm) | | 10.3 mm) | |
| | # | 320 | 330 | 360 | 360 | Delta | 360 | Delta |
| 5 mL | 1 | 5.02 | 22.840 | 6.37 | 6.84 | 0.47 | 7.54 | 1.17 |
| | 2 | 5.29 | 23.070 | 6.48 | 7.07 | 0.59 | 7.77 | 1.29 |
| | 3 | 4.91 | 22.290 | 7.08 | 6.29 | −0.79 | 6.99 | −0.09 |
| | 4 | 5.18 | 22.600 | 6.71 | 6.60 | −0.11 | 7.30 | 0.59 |
| | 5 | 5.22 | 22.820 | 6.57 | 6.82 | 0.25 | 7.52 | 0.95 |
| Average | | 5.12 | 22.724 | 6.64 | 6.72 | | 7.42 | |
| Stdev | | 0.14 | 0.263 | 0.25 | 0.26 | | 0.26 | |
| 3.5 mL | 1 | 5.55 | 36.410 | 19.09 | 20.41 | 1.32 | 21.11 | 2.02 |
| | 2 | 5.09 | 35.590 | 19.41 | 19.59 | 0.18 | 20.29 | 0.88 |
| | 3 | 4.97 | 36.160 | 20.03 | 20.16 | 0.13 | 20.86 | 0.83 |
| | 4 | 4.98 | 35.760 | 19.79 | 19.76 | −0.03 | 20.46 | 0.67 |
| | 5 | 5.08 | 35.810 | 19.44 | 19.81 | 0.37 | 20.51 | 1.07 |

| | | | | | |
|---|---|---|---|---|---|
| Average | 5.13 | 35.946 | 19.55 | 19.95 | 20.65 |
| Stdev | 0.21 | 0.297 | 0.33 | 0.30 | 0.30 |

The proposed stopper height position 360 for stopper 200, 400, 450 placement is:

```
Stopper height position 360 = average (top syringe flange to liquid
surface 330) − (head space 320 + stopper length 340)
  Stopper height (5.0 mL) = 22.72 − (5+11.0) = 6.724 mm → 6.7 mm
  Stopper height (3.5 mL) = 35.95 − (5+11.0) = 19.946 mm → 19.9 mm
```

### Example 2: Stopper Height Values and a in Process Control Method

In one exemplary embodiment, an IPC method is provided for a medicament, such as paliperidone palmitate equivalent to about 200 mg/mL extended release suspension for injection intended as a 6 month dose, which may be used to treat schizophrenia, and a syringe filling process for the same. Also, a process for determining optimal stopper height values for the IPC method is provided.

1. Objective: The development of paliperidone palmitate equivalent to about 200 mg/mL extended release (6 month) suspension for injection included development of syringe filling formats. Stopper height values were determined for the syringe filling formats. The IPC method of stopper height determination may be performed by a digital depth meter, such as Mitutoyo, code 543-250B. A set point for the instrument which performs the stopper height position is determined using the IPC method. Paliperidone palmitate may be administered in the following presentations: about 3.5 mL and about 5 mL or any other suitable presentation.

TABLE 2
Filling Volumes, PP6M

| | Volume (mL) | |
|---|---|---|
| | 3.5 mL | 5.0 mL |
| Upper Action Limit | 3.704 | 5.229 |
| Target | 3.617 | 5.104 |
| Lower Action Limit | 3.529 | 4.979 |

TABLE 3
Filling Volumes, PP6M

| | Weight (g) | |
|---|---|---|
| Density = 1.064 g/mL | 3.5 mL | 5.0 mL |
| Upper Action Limit | 3.941 | 5.564 |
| Target | 3.848 | 5.431 |
| Lower Action Limit | 3.755 | 5.298 |

TABLE 4
Stopper Positions

| | | | |
|---|---|---|---|
| Volume | H = target | Lower | Upper |
| (mL) | (camera) | Specification | Specification |

PP6M (Reg./PPQ)

| | | | |
|---|---|---|---|
| 3.5 | 19.9 | 18.41 | 21.39 |
| 5.0 | 6.7 | 5.21 | 8.19 |

2. Definitions: The following materials were used in the exemplary process:
Schott COC syringe 5 mL (used for both 3.5 mL and 5 mL presentation)
Height of syringe (with tip cap): 84.2-85.5 mm
Height of barrel: 76.7-77.1 mm
Internal diameter: 12.1-12.2 mm
Datwyler 5 mL stopper:
Stopper Height: 9.8-10.8 mm
External diameter: 12.5-12.9 mm

As shown in FIGS. 5A and 5B, the stopper height H in a paliperidone palmitate pre-filled syringe 100 intended as a 6 month dose may be determined by the difference between the syringe flange F (bottom side) to the stopper lower edge (the edge closer to the syringe tip). The stoppers 400 for an exemplary 1 mL syringe (FIG. 4A) have a conical tip 410 and the stopper edge 420 is proximal to the tip. The stopper height H is measured to the stopper edge 420 (not to the conical tip 410). The examples shown in FIGS. 5A and 5B are for illustrative purposes only. The size of the syringe (here, 1 mL) is exemplary. FIGS. 5A and 5B provide an exemplary procedure for determining a stopper height, regardless of the size of the syringe.

A digital depth meter 500 is provided with a stainless steel reference block 520 (FIGS. 5A and 5B). A rod 530 protruding from the depth meter is completely extended out in a rest condition (FIG. 5A, device 500 before inserting into the syringe (i.e., the device 500 is at rest)). When the syringe 100 is placed against the block 520, the rod 530 is displaced by the stopper (e.g., 200, 400, 450) and a display 510 shows the distance of displacement of the rod 530 relative to the rest condition (FIG. 5B, device 500 during measurement). The readout or display 510 of the instrument or meter 500 gives the following values (mm):

$$\begin{matrix}( 2. a ) & \\ \begin{matrix}RDT = R - ( H + F ) & for the stoppers for 1. mL syringes , and\end{matrix} & ( FIG . 4 A )\end{matrix}$$

$$\begin{matrix}( 2. b ) & \\ \begin{matrix}RDT = R - ( H + F ) + C & for the stoppers for the 2.25 mL and 2.8 mL syringes ,\end{matrix} & ( FIG . 4 B )\end{matrix}$$

where, RDT is Readout; R is Rest position length; H is Stopper height; F is Flange thickness; C is a correcting factor for the stopper 400 having the conical tip 410 due to misalignment between the bottom of the inner screw and the stopper edge 420=1.7 mm (FIG. 4A).

3. Stoppering height calculation method: The stopper height H is calculated based on the allowed head space between the product 10 and the stopper 200, 400, 450.

4. Product height: The product height was measured by filling water in PalPal syringes (i.e., depending on filling volume, sizes 1 mL Long, 2.25 mL; 2.8 mL and 5 mL syringe) according to the following procedure:

Fill a 22 gauge-10 mL syringe with water and carefully remove the air.

Place an empty PalPal syringe on the syringe holder previously placed on the IPC balance and tare.

Dose an appropriate amount of water verifying the weight on the balance.

If the weight deviates from the desired value extract or add product in order to obtain the desired weight.

Remove the syringe from the holder and make sure no drops of water are sticking on the syringe wall.

Measure the product height by means of a caliper, e.g., Mitutoyo CD-15DCX.

5. Stopper height: The stopper height target (H) is determined as the stopper 200, 400, 450 positioned above the product height by the distance equal to the head space (target about 5 mm or about 6 mm, see above). This is: H=product height−head space.

6. Stopper height position IPC method: All values in relationship (2.a) and (2.b) are defined before experimentation. Therefore, the instrument set point is determined during IPC of stopper height.

7. Conclusion: The stopper height IPC of paliperidone palmitate filling process is performed measuring the stopper height by means of the digital depth meter Mitutoyo (code 543-250B) shown in FIGS. 5A and 5B. The value may be set on the instrument with a tolerance of ±1 mm.

### Example 3: Head Space Trials for PP6M

Relatively smaller head spaces, e.g., about 2 mm and about 4 mm, were evaluated. Results were generally less favorable than those observed when head space was about 5 mm or about 6 mm.

Purpose: Provide water-filled syringes of two stopper designs with two different head spaces to support stopper movement testing. Head spaces of about 2 mm and about 4 mm were evaluated. Also, evaluate processing of stoppers on a filling/stoppering line of a manufacturing facility. Further, test different head spaces for product filled syringes for manufacturability.

Referring to the photograph of FIG. 6, two measurements were performed: a measurement from a top of a flange 630 of a syringe to a top of a stopper 620 (similar to the stopper height position 360 from top of flange to top of stopper shown in FIG. 3), and a measurement from the top of the flange of the syringe to a top of a liquid 610 in the syringe (similar to height from top of syringe to liquid surface 330 shown in FIG. 3).

Results (general, water-filled samples): with about 4 mm head space, no problems during stoppering, filling OK. However, with about 2 mm head space, on several positions the insertion tube touched the liquid surface resulting in water on top of the stopper after placement. The most probable root cause: variation in liquid column height/filling volume and low head space. The risks associated with improper head space include: rejections during optical inspection (a contaminated insertion tube carries over product to subsequently filled syringes, which, if not noticed in time, may result in a high rejection rate); and aseptic intervention required in an isolator of the manufacturing facility.

TABLE 5
Results of Experimental Trials

| | Liquid to flange | Stopper to flange | Stopper height | Head space |
|---|---|---|---|---|
| Tub3 | 23.55 | 10.37 | 10.2 | 2.98 |
| | 23.7 | 10.68 | 10.2 | 2.82 |
| | 23.25 | 10.59 | 10.2 | 2.46 |
| | 23.65 | 10.42 | 10.2 | 3.03 |
| Tub4 | 23.1 | 9.29 | 10.2 | 3.61 |
| | 22.9 | 9.58 | 10.2 | 3.12 |
| | 22.8 | 9.7 | 10.2 | 2.9 |
| Tub5 | 23.45 | 8.48 | 10.2 | 4.77 |
| | 23.15 | 8.96 | 10.2 | 3.99 |
| | 22.9 | 8.3 | 10.2 | 4.4 |
| | 22.9 | 8.28 | 10.2 | 4.42 |
| Tub6 | 23.2 | 8.39 | 10.2 | 4.61 |
| | 23.5 | 8.8 | 10.2 | 4.5 |
| | 23.15 | 8.39 | 10.2 | 4.56 |
| | 22.9 | 8.2 | 10.2 | 4.5 |
| | 22.4 | 8.21 | 10.2 | 3.99 |
| Tub7 | 23.5 | 8.68 | 10.2 | 4.62 |
| | 23.3 | 8.44 | 10.2 | 4.66 |
| Tub10 | 23.3 | 10.39 | 10.2 | 2.71 |
| | 23.15 | 10.26 | 10.2 | 2.69 |
| | 23.2 | 10.79 | 10.2 | 2.21 |
| | 22.95 | 10.42 | 10.2 | 2.33 |
| Tub11 | 23.3 | 11.16 | 10.2 | 1.94 |
| | 23.3 | 10.53 | 10.2 | 2.57 |
| | 23.2 | 10.77 | 10.2 | 2.23 |
| Tub14 | 23 | 10.1 | 10.3 | 2.6 |
| | 23.6 | 10.44 | 10.3 | 2.86 |
| | 23.2 | 10.36 | 10.3 | 2.54 |
| | 23.1 | 9.97 | 10.3 | 2.83 |
| Tub15 | 23.5 | 10.77 | 10.3 | 2.43 |
| | 22.95 | 9.77 | 10.3 | 2.88 |
| | 23.2 | 10.3 | 10.3 | 2.6 |
| | 23.1 | 10.14 | 10.3 | 2.66 |
| Tub18 | 23.05 | 8.7 | 10.3 | 4.05 |
| | 22.8 | 8.38 | 10.3 | 4.12 |
| | 22.75 | 8.44 | 10.3 | 4.01 |

A REF-stopper was used for Tub3, Tub4, Tub5, Tub6, Tub7, Tub10 and Tub11. For Tub3, Tub4, Tub5 and Tub6, a stopper height setting was about 4 mm. For Tub7, a head space was about 5 mm. For Tub10, a stopper height setting was about 2 mm. For Tub11, a head space was about 2 mm.

A P8790TR-stopper was used for Tub14, Tub15 and Tub18. For Tub14, a stopper height setting was about 2 mm. For Tub15, a head space was about 2 mm. Also, for Tub14 and Tub15, water was observed on several stoppers. For Tub18, a head space was about 4 mm.

FIG. 7A is a photo of a filled syringe using the P8790TR-stopper with a stopper height setting of about 2 mm. FIG. 7B is a photo of a filled syringe using the P8790TR-stopper with a stopper height setting of about 3 mm.

As shown in FIG. 8A, a pre-filled syringe assembly 100 includes a cannula or needle assembly 110, which is removably attached to a distal end of a syringe barrel 120. In an exemplary embodiment, the pre-filled syringe assembly 100 is configured for attachment to any one of an 18 gauge, 19 gauge, 20 gauge, 21 gauge, 22 gauge and 23 gauge cannula thin walled cannula or needle. In some exemplary embodiments, the reconstituted medicament may be relatively difficult to inject into a patient in need. The 20 gauge or 22 gauge thin walled cannula or needle achieves a specific and desirable feel when a medical professional presses on the plunger. The 20 gauge or 22 gauge thin walled cannula or needle achieves a desirable amount of resistance, i.e., not too hard to move the plunger while also not causing the plunger to move too fast. The 20 gauge or 22 gauge thin walled cannula or needle provides a desirable amount of back pressure when the medicament is being delivered to the patient.

In some embodiments, the thin walled cannula or needle may be a Medtronic needle having an outside diameter of about 0.0355 mm to about 0.0360 mm, an inside diameter of about 0.0260 mm to about 0.0275 mm, and a free length of needle of about 1.455 in (about 36.96 mm) to about 1.525 in (about 38.74 mm). Although illustrated in FIG. 8A, it is understood that the needle assembly 110 need not be attached during filling of the pre-filled syringe assembly 100, or during shipping or storage of the pre-filled syringe. The needle is typically attached to the syringe prior to the need for an injection, and such attachment may be via a Luer connection as shown in FIG. 1A and described below.

As shown in FIG. 8B, the syringe barrel 120 is filled with a liquid or medicament 10, and the liquid or medicament 10 has a fill volume 150 measured from a distal end of the syringe body to a top surface of the liquid or medicament 10. The liquid or medicament 10 is measured from a bottom of the meniscus when the liquid or medicament 10 forms a meniscus concave inward toward the distal end. Once the syringe is filled with a medicament, the stopper is placed in the syringe with a temporary sleeve 130. In such a process, the sleeve 130 is inserted into a proximal end of the syringe barrel 120, and the sleeve 130 is provided at a height 170 measured from a bottom of the sleeve 130 to a bottom of a flange of the syringe barrel 120. The stopper 200 is inserted into a proximal end of the sleeve 130 with a temporary push rod 140 to a desired position that provides an optimal head space. Head space 160 is the distance between a distal end of the sleeve 130 and a top surface of the liquid or medicament 10.

FIGS. 8A to 8F, inclusive, illustrate a vented tubes stoppering principle. In FIG. 8A, the needle assembly 110 is attached to a distal end of the syringe barrel 120. The syringe barrel 120 is filled with the liquid or medicament 10. In FIG. 8B, a sleeve 130 is inserted into a proximal end of the syringe barrel 120, and the sleeve 130 is provided at a height 170 measured from a bottom of the sleeve 130 to a bottom of a flange. A stopper 200 is inserted into a proximal end of the sleeve 130 with a temporary push rod 140. In FIG. 8C, the temporary push rod 140 pushes the stopper 200 to a distal end of the sleeve 130. In FIG. 8D, the sleeve 130 is removed. In FIG. 8E, the temporary push road 140 is removed leaving the stopper 200 inside the syringe barrel 120 at the height 170. In FIG. 8F, a height 175 is measured from a proximal end of the stopper 200 to the bottom of the flange.

Critical points regarding vented tubes stoppering include the variation of height 170, variation of fill volume/column height, and target head space. Specifically, the variation of height 170 is ±1 mm. The variation of fill volume/column height between lower and upper limits (−T2 to +T2) for a 3.5 mL presentation is ±0.75 mm; and for a 5.0 mL presentation, ±1.05 mm. The target head space is sufficient to allow air to escape from the stopper 200. Also, the closer the tube to the liquid surface, the higher the risk will be that flow will push out liquid. The lower the head space, the higher the risk of contact between the tube and liquid due to variation in the height 170 and fill volume. Also, liquid between the stopper and the syringe may result in increased rejections during optical inspection. Since the stoppers are vented, air can escape from the syringe during filling and insertion of the stopper is facilitated.

### Example 4: P8790TR-Stopper with Overfill Syringes, 5 mL Presentation

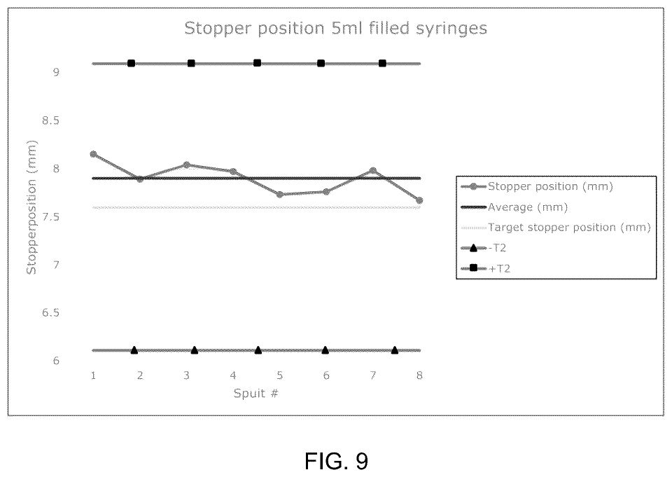

Table 6 reports results of experimental trials utilizing the P8790TR-stopper with overfill syringes in the 5 mL presentation. The results in Table 6 are graphed in FIG. 9. Photographs from each of Trials 1-8, inclusive, are provided at FIGS. 10A to 10H, inclusive, respectively. The photographs were obtained with a digital camera. The images may be used to confirm that a given head space is achieved.

TABLE 6
Results, P8790TR-stopper with Overfill Syringes, 5 mL
Presentation stopper

| | Stopper position | Target stopper | | |
|---|---|---|---|---|
| Trial # | (mm) | position (mm) | −T2 | +T2 |
| 1 | 8.15 | 7.6 | 6.11 | 9.09 |
| 2 | 7.89 | 7.6 | 6.11 | 9.09 |
| 3 | 8.04 | 7.6 | 6.11 | 9.09 |
| 4 | 7.97 | 7.6 | 6.11 | 9.09 |
| 5 | 7.73 | 7.6 | 6.11 | 9.09 |
| 6 | 7.76 | 7.6 | 6.11 | 9.09 |
| 7 | 7.98 | 7.6 | 6.11 | 9.09 |
| 8 | 7.67 | 7.6 | 6.11 | 9.09 |
| Average | 7.90 | | | |
| Delta | 0.30 | | | |

The above-referenced examples confirm that a head space of greater than about 3 mm and less than about 7 mm minimized both frothing and clogging problems. In some embodiments, the head space is greater than about 4 mm and less than about 6 mm. In some embodiments, the head space is about 4 mm. In some embodiments, the head space is about 5 mm. As noted above, too much head space may contribute to excess and undesirable frothing of the reconstituted drug suspension; whereas, too little head space may contribute to improper mixing of the drug suspension, and thus the presence of relatively large particles in the suspension, which can clog the delivery cannula.

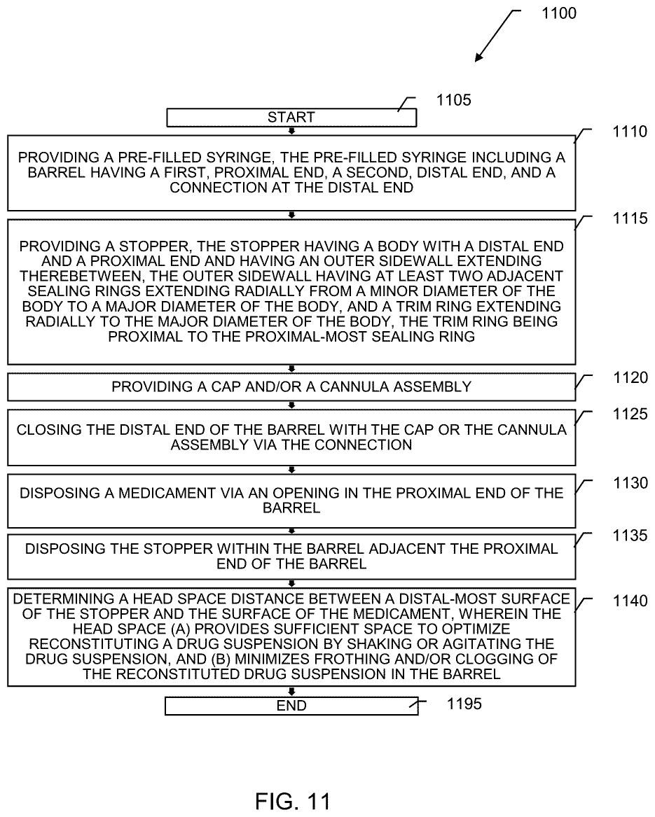

FIG. 11 is a flow chart illustrating steps of a method of providing a pre-filled syringe according to an exemplary embodiment. A method 1100 may include one or more steps. The method 1100 may include a start 1105 and an end 1195. The method 1100 may include a step 1110 of providing a pre-filled syringe, the pre-filled syringe including a barrel having a first, proximal end, a second, distal end, and a connection at the distal end. The method 1100 may include a step 1115 of providing a stopper, the stopper having a body with a distal end and a proximal end and having an outer sidewall extending therebetween, the outer sidewall having at least two adjacent sealing rings extending radially from a minor diameter of the body to a major diameter of the body, and a trim ring extending radially to the major diameter of the body, the trim ring being proximal to the proximal-most sealing ring. The method 1100 may include a step 1120 of providing a cap and/or a cannula assembly. The method 1100 may include a step 1125 of closing the distal end of the barrel with the cap or the cannula assembly via the connection. The method 1100 may include a step 1130 of disposing a medicament via an opening in the proximal end of the barrel. The method 1100 may include a step 1135 of disposing the stopper within the barrel adjacent the proximal end of the barrel. The method 1100 may include a step 1140 of determining a distance from a proximal-most surface of the syringe to a surface of the medicament less the sum of a length of the stopper and a head space distance between a distal-most surface of the stopper and the surface of the medicament.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in kits, systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A pre-filled syringe, comprising:
a barrel having a first, proximal end and a second, distal end with a medicament disposed therein, wherein the medicament is a suspension of paliperidone palmitate, and the syringe has a nominal capacity of 5 mL; and
a stopper disposed within the barrel adjacent the first end of the barrel, the stopper having a body with a distal end and a proximal end and having an outer sidewall extending therebetween, the outer sidewall having at least two adjacent sealing rings extending radially from a minor diameter of the body to a major diameter of the body,
wherein for a presentation of the medicament having a volume of about 3.5 mL, the ratio of a target head space distance between a distal-most surface of the stopper and the surface of the medicament to the target volume of the presentation of the medicament is greater than about 0.86 mm/mL and less than about 2.00 mm/mL,
wherein for a presentation of the medicament having a volume of about 5 mL, the ratio of the target head space distance between a distal-most surface of the stopper and the surface of the medicament to the target volume of the presentation of the medicament is greater than about 0.60 mm/mL and less than about 1.40 mm/mL, and,
wherein the target head space distance between a distal-most surface of the stopper and the surface of the medicament (a) provides sufficient space to optimize reconstituting a drug suspension by shaking or agitating the drug suspension, and (b) minimizes frothing and/or clogging of the reconstituted drug suspension in the barrel.
